(19) United States
(12) Patent Application Publication
(10) Pub. No.: US 2013/0150674 A1
(43) Pub. Date: Jun. 13, 2013

(54) THORACIC SCOPE PORT SPONGE CLEANER

(71) Applicant: Covidien LP, Mansfield, MA (US)

(72) Inventors: Fiona Middlemiss Haig, Boston, MA (US); Cormac O'Prey, Hertfordshire (GB)

(73) Assignee: COVIDIEN LP, Mansfield, MA (US)

(21) Appl. No.: 13/706,601

(22) Filed: Dec. 6, 2012

Related U.S. Application Data
(60) Provisional application No. 61/567,878, filed on Dec. 7, 2011.

(51) Int. Cl. A61B 1/00

(52) U.S. Cl. CPC A61B 1/00131
USPC 600/155

(57) ABSTRACT

An instrument for cleaning a lens of a scope includes an elongated sheath and a cleaner. The elongated sheath defines a lumen dimensioned and configured to slidably receive the scope therethrough. The cleaner is positioned within the sheath at a distal end portion of the lumen. The cleaner includes a fluid receiving portion configured to clean the lens of the scope in response to translation of the scope therethrough.

### CROSS-REFERENCE TO RELATED APPLICATIONS

This application claims priority to, and the benefit of, U.S. Provisional Patent Application Ser. No. 61/567,878, filed on Dec. 7, 2011, the entire contents of which are incorporated by reference herein.

### BACKGROUND

1. Technical Field

The present disclosure relates to a cleaning apparatus configured to remove debris from the lens of a minimally invasive viewing instrument.

2. Background of Related Art

Minimally invasive surgery has become increasingly popular in recent years. Minimally invasive surgery eliminates the need to cut a large incision in a patient, thereby reducing discomfort, recovery time, and many of the deleterious side effects associated with traditional open surgery. Minimally invasive viewing instruments, e.g., laparoscopes and endoscopes, are optic instruments to facilitate the viewing of internal tissues and/or organs.

Laparoscopic surgery involves the placement of a laparoscope in a small incision in the abdominal wall of a patient to view the surgical site. Endoscopic surgery involves the placement of an endoscope in a naturally occurring orifice, e.g., mouth, nose, anus, urethra, and vagina to view the surgical site. Other minimally invasive surgical procedures include video assisted thoracic surgery and cardiovascular surgery conducted through small incisions between the ribs. These procedures also utilize scopes to view the surgical site.

A typical minimally invasive viewing instrument, e.g., a laparoscope or an endoscope, includes a housing, an elongated lens shaft extending from one end of the housing, and a lens that is provided in the distal end of the lens shaft. A camera viewfinder extends from the other end of the housing. A camera is connected to the housing and transmits images of the surgical field viewed through the lens to a monitor on which the images are displayed. During a surgical procedure, the distal end portion of the lens shaft is extended into the patient, while the proximal end portion of the lens shaft, the housing and the camera viewfinder remain outside the patient. In this manner, the laparoscope/endoscope is positioned and adjusted to view particular anatomical structures in the surgical field on the monitor.

During insertion of an endoscope or a laparoscope into the body and during the surgical procedure, debris, e.g., organic matter and moisture, may be deposited on the lens of the endoscope. The buildup of debris and condensation on the lens impairs visualization of the surgical site, and often necessitates cleaning of the lens.

### SUMMARY

The present disclosure is generally related to an instrument for cleaning the lens of a medical viewing instrument, such as an endoscope, during a minimally invasive surgical procedure.

In one aspect, the disclosure provides an instrument for cleaning a lens of a scope including an elongated sheath having a proximal portion and a distal portion. The sheath defines a lumen extending longitudinally therethrough. The lumen is dimensioned and configured to slidingly receive the scope therein. A cleaner is positioned within the lumen of the sheath at a distal portion and has a fluid receiving portion. The fluid receiving portion is configured to clean the scope as the scope passes through the cleaner.

In some embodiments, the cleaner obstructs the lumen of the sheath in a first position and the cleaner transitions toward a second position in response to the axial translation of the scope through the cleaner. The cleaner in some embodiments is biased toward the first position.

The cleaner may be formed from a compressible material. As the viewing instrument (scope) axially translates through the distal portion of the sheath, the scope passes across or through the cleaner and compresses the cleaner. The compression of the cleaner can both clean the lens and facilitate the translation of the scope through the distal portion of the sheath. The compressible material can include a sponge-like material.

The cleaner may also include a guide channel. The guide channel may be centrally and longitudinally disposed within the cleaner to guide the scope through and/or by the cleaner. In response to the axial translation of the scope through the distal portion of the sheath, the diameter of the guide channel may expand. The guide channel may be biased toward a state in which the guide channel is substantially closed to facilitate a continuous contact between the cleaner and the lens as the scope translates through the guide channel. The sliding contact between the lens and the surfaces of the guide channel of the cleaner facilitates the removal of debris and/or moisture from the lens of the scope.

In addition, a fluid conduit may be provided in fluid communication with the scope/lens and/or the cleaner. In particular, the fluid conduit may include an outlet that discharges or ejects a fluid or a gel onto or into the cleaner so that as the scope translates through the distal portion of the sheath, the lens comes into contact with the fluid or gel. Continued translation of the scope through the cleaner can wipe and/or dry the fluid or gel from the surface of the viewing portion.

These and other features of the present disclosure will be more fully described with reference to the appended figures.

### BRIEF DESCRIPTION OF THE DRAWINGS

By way of description only, embodiments of the present disclosure will be described herein with reference to the accompanying drawings, in which:

FIG. 1 is a schematic view of a scope lens cleaner according to an embodiment of the present disclosure;

FIG. 2 is a cross-sectional view, taken along section line 2-2 of FIG. 1;

FIG. 3 is an enlarged, cross-sectional view of a distal end of the instrument of FIG. 1;

FIG. 4A is an enlarged, perspective view of a distal end of the instrument of FIG. 1 shown with a scope in a first, retracted state;

FIG. 4B is an enlarged, perspective view of the distal end of the instrument of FIG. 1 shown with a scope in an intermediate state; and

FIG. 4C is an enlarged, perspective view of the distal end of the instrument of FIG. 1 shown with a scope in a deployed (advanced) state.

### DETAILED DESCRIPTION

Particular embodiments of the present disclosure will be described with reference to the accompanying drawings. In the figures and in the description that follow, in which like reference numerals identify similar or identical elements, the term “proximal” will refer to the end of the device that is closer to the operator during use, while the term “distal” will refer to the end that is farther from the operator during use.

An endoscope typically includes an endoscope housing or body which can be rigid or flexible, depending on its surgical application. A camera viewfinder, e.g. an eyepiece, is located at a proximal (imaging) end of the scope housing. A lens is provided at the distal end of the scope body.

In typical use of the endoscope, the viewfinder is adapted to sight images of a surgical field in the patient, e.g. an abdominal cavity, thoracic cavity, etc., as the position of the scope is adjusted to view a particular anatomical structure or structures in the surgical field. The camera is adapted to receive images of the surgical field sighted through the lens and transmit the images to an external monitor that is connected to the camera and on which the images of the surgical field are displayed. That is, a visual display device is operatively connected to the eyepiece to convert the optical signal into a video signal to produce a video image on the monitor (or for storage on select media). Accordingly, the monitor enables a surgical team to view the anatomical structure or structures in the surgical field inside the patient as the surgical procedure is carried out using minimally invasive or endoscopic surgical instruments. Throughout the surgical procedure, condensation, smoke particles, biological tissue or matter has a tendency to contact and build up on the lens of the scope. This tends to obscure the images of the surgical field as they are displayed on the monitor.

The instrument of the present disclosure enables cleaning of the scope lens during the surgical procedure to maintain a clear image without having to remove the scope from the patient's body.

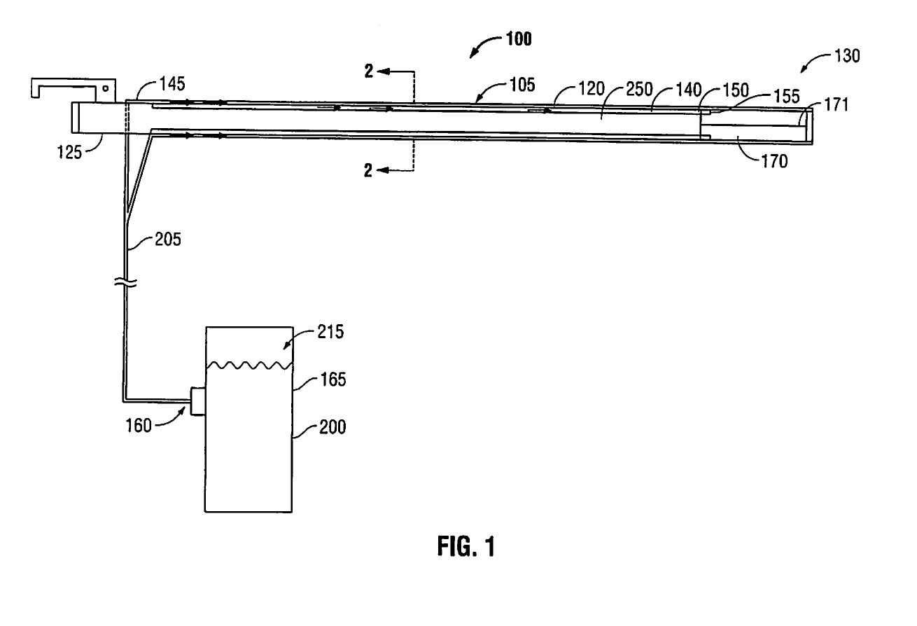

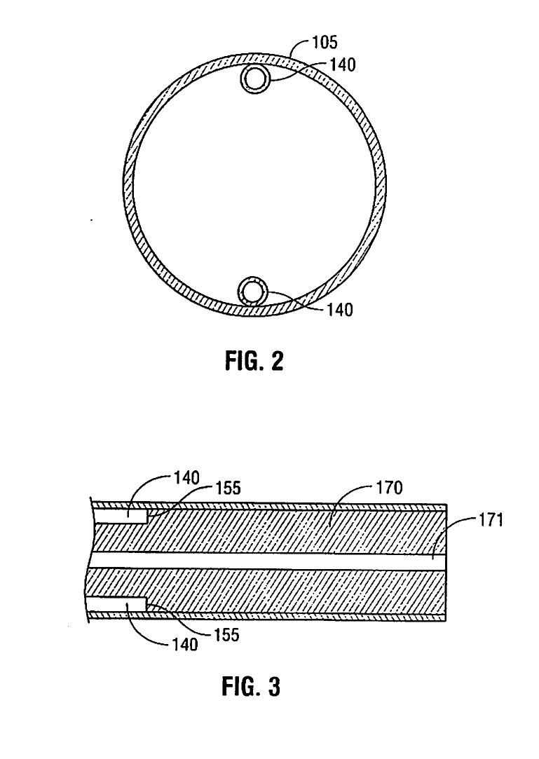

A minimally invasive surgical instrument 100 that is configured and adapted to clean a viewing portion 265, e.g., a lens, of a viewing instrument or scope 250 disposed within surgical instrument 100 will now be described with reference to FIGS. 1-4C. The surgical instrument 100 generally includes an elongated cylindrical or tubular sheath 105 defining a longitudinally extending lumen 109 along a longitudinal axis. The sheath 105 may have a generally annular or circular cross-section as is shown in FIGS. 1-4C. However, in other embodiments, the sheath 105 may have a non-circular cross-section. The viewing instrument 250 is positioned within the sheath 105 and is configured and adapted to axially translate a distance through the lumen of sheath 105.

The tubular sheath 105 is dimensioned and configured to slidingly receive a conventional scope therein. The scope can be inserted into an already placed sheath or alternatively positioned within the sheath and together inserted into the body. The scope can be fully removed from the sheath if desired. The sheath can accommodate various types of scopes, including but not limited to laparoscopes, thoracic scopes, etc. For example, during video assisted thorascopic surgery, a thoracic port is inserted through the ribs to provide access to the thoracic cavity for access to the lung or other tissue. A separate access is provided through the ribs to insert a scope to visualize the thoracic cavity during the surgical procedure. The sheath of the present disclosure can be utilized with the thoracic scope to clean the lens with minimal disruption to the surgical procedure, thereby improving visibility and imaging during the surgical procedure. The sheath can also be utilized with a flexible scope if composed of a sufficiently flexible material.

One or more fluid conduits 140 may be coupled to the sheath 105. Two conduits 140 are shown in the embodiment of FIG. 1, one on each side of tubular sheath 105, although they can be in other positions with respect to the sheath 105. The fluid conduits 140 each include a proximal portion 145 and a distal portion 150. The distal ends 150 of the fluid conduits 140 include outlets or openings 155. Each outlet 155 abuts or is adjacent to a proximal end of a compressible cleaner 170 to moisten the compressible cleaner 170 with a cleaning fluid 200. The outlet(s) 155 may also be directed toward a surface of the viewing portion 265. The outlet 155 can in some embodiments include a fluid discharge nozzle (not shown). The compressible cleaner 170 in one embodiment is composed of a sponge-like material. Alternatively, it could be a toweling/absorbent textile. The material can be deformed as the tip of the scope is torqued against the material for cleaning. The compressible cleaner may have a sufficient length to facilitate a gradient in which the proximal end of the cleaner 170 is wetter, or moister, than the distal end of the cleaner 170 such that during translation of the scope 250 the viewing portion 265 is first moistened and then is dried during the axial translation of the scope 250 through the sheath 105.

The proximal ends 145 of the fluid conduits 140 are coupled to a proximal end 125 of the sheath 105. The proximal end portions 145 of the fluid conduits are coupled to a connecting conduit 205 which has an inlet 160. The inlet 160 is in fluid communication with a fluid pump and supply apparatus 215. The fluid pump and supply apparatus 215 include a fluid reservoir 165 containing a fluid 200, i.e., a cleaning solution. The fluid 200 may be, but is not limited to, a saline or a gel solution. The fluid 200 may have particular chemical properties to facilitate cleaning and/or rapid drying of the viewing portion 265 subsequent to its application on to the viewing portion 265. It is also contemplated that different fluids may be use in sequence as part of a wash/rinse/dry cycle.

The fluid conduits 140 provided on each side of the tubular sheath 105 have a generally annular cross-sectional configuration. The fluid conduits 140 are connected along the sheath exterior 120 and extend generally parallel to the longitudinal axis of the tubular sheath 105. The diameter or width of the fluid conduits 140 may in some embodiments be substantially equal to or slightly larger than the thickness of the sheath wall. As shown, two substantially identical fluid conduits 140 are provided. Conduits 140 are spaced apart about 180 degrees. However, other spacings are also contemplated. A different number of fluid conduits could also be provided. If a flexible sheath is utilized for a flexible scope, the fluid conduits would also be composed of sufficiently flexible material.

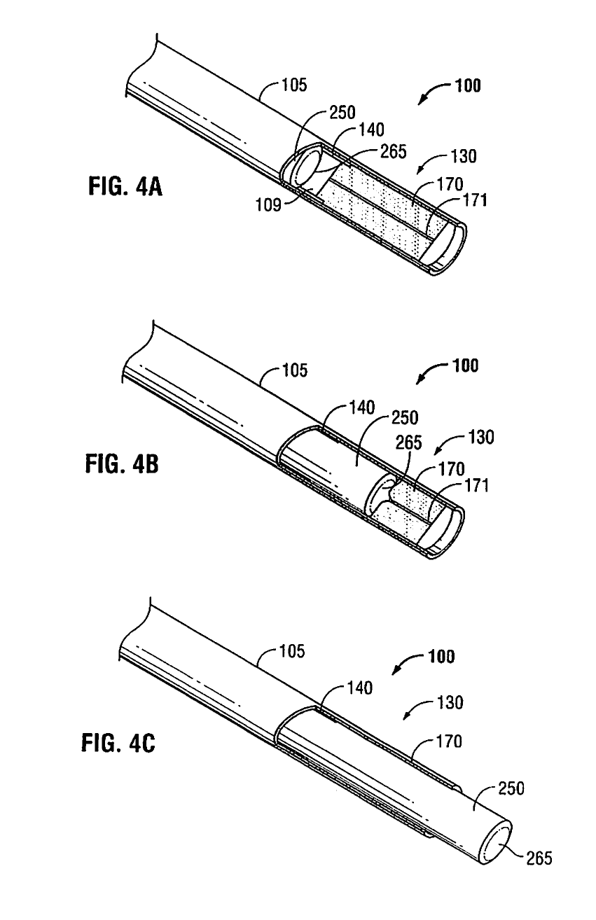

The compressible cleaner 170 includes a guide channel 171 extending therethrough and is positioned at a distal end portion 130 of surgical instrument 100. The compressible cleaner 170 is generally cylindrical and includes guide channel 171 disposed along a central longitudinal axis. The scope 250 is configured and adapted to axially translate through the guide channel 171 defined by the compressible cleaner 170. The guide channel 171 is substantially closed in an initial position (FIG. 4A) to facilitate full contact with the viewing portion 265 of the scope 250 during the distal translation of the scope 250 through the guide channel 170. The compressible cleaner 170 is biased toward the initial position in which the guide channel 171 is substantially closed, i.e., the diameter of the guide channel 171 has a near zero or negligible dimension. It is also contemplated that the guide channel may include a series of radial slits defining, e.g. a star pattern, to enable an expansion thereof.

While the compressible cleaner 170 is shown and described as having a cylindrical shape defining a central, longitudinally extending guide channel 171, other configurations of compressible cleaner 170 are within the scope and spirit of the present disclosure. In particular, the compressible cleaner 170 may be devoid of a central guide channel 171. In such an embodiment, a portion of the compressible cleaner 170 may be secured to the interior of the sheath 105 and an opposing portion may be detached from the interior of the sheath 105 such that upon translation of the viewing instrument 250 through sheath 105, the compressible cleaner 170 compresses toward the portion of the cleaner 170 secured to the interior of the sheath 105. Moreover, in still further embodiments, multiple compressible cleaners may be secured to the interior of the sheath 105 and arranged to form a common guide channel for the translation of the scope 250 therethrough. Common to each embodiment is the translation of a surface or surfaces of the compressible cleaner 170 against the viewing portion 265 of the scope 250 to facilitate removal of contaminants, e.g., debris and/or moisture, from the viewing portion 265.

The use and operation of the compressible cleaner 170 will now be described with reference to FIGS. 4A-4C. As described above, the compressible cleaner 170 is disposed at the distal end portion 130 of the sheath 105. With the scope 250 in an initial, retracted position within the sheath 105, the fluid 200 may be ejected from the outlet openings 155 of the fluid conduits 140. As described above, the outlet openings 155 abut or are adjacent the cleaner 170. The cleaner 170 may have absorbable properties such that the cleaner 170 is moistened by the fluid 200. Alternatively, the outlet openings 155 may be directed toward the viewing portion 265 of the scope 250. When the scope 250 is retracted in the initial position, as shown in FIG. 4A, there may be a gap between the viewing portion 265 and the proximal end of the cleaner 170. As the scope 250 is distally translated through the sheath 105, the viewing portion 265 contacts the cleaner 170 and parts the cleaner 170 by expanding the dimension of the guide channel 171 (FIG. 4B). In the fully extended position, as shown in FIG. 4C, the scope 250 is fully extended through the channel 171 and out of the sheath 105, unobstructed by the cleaner 170. When the lens becomes soiled, the scope 250 is proximally retracted at least partially through the cleaner 170. Optionally, fluid 200 may be ejected through the fluid conduits 140 as described above to apply fluid 200 to the compressible material 170 to add more moisture and/or to the viewing portion 265. The cleaner 170 may both moisten and dry the viewing portion 265. The repeated proximal and distal axial translation through the cleaner 170 effects cleaning of the viewing portion 265 of the viewing instrument.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An instrument for cleaning a lens of a scope, comprising:
an elongated sheath having a proximal portion and a distal portion, the sheath defining a lumen extending longitudinally therethrough, the lumen dimensioned and configured to slidingly receive the scope therein;
a cleaner positioned within the lumen of the sheath at the distal portion, the cleaner having a fluid receiving portion configured to clean the lens of the scope as the scope passes through the cleaner; and
a fluid conduit connected to the sheath for transporting fluid and having a discharge opening adjacent the fluid receiving portion of the cleaner to provide the fluid to the cleaner.

2. The instrument of claim 1, wherein the cleaner substantially obstructs the lumen of the sheath in a first position, the cleaner transitionable to a second position upon axial movement of the scope through the cleaner.

3. The instrument of claim 2, wherein the cleaner is biased toward the first position.

4. The instrument of claim 1, wherein the cleaner is composed of a compressible sponge-like material.

5. The instrument of claim 4, wherein the compressible sponge-like material includes a cleaning gel.

6. The instrument of claim 4, wherein the compressible sponge-like material includes a cleaning saline.

7. The instrument of claim 1, wherein the cleaner includes a guide channel extending longitudinally therethrough, the channel dimensioned to receive the scope therethrough.

8. The instrument of claim 7, wherein the guide channel defines a diameter, the diameter of the guide channel expanding in response to translation of the scope through the guide channel, the guide channel biased toward a substantially closed diameter.

9. The instrument of claim 8, wherein axial movement of the scope through the guide channel brings the lens of the scope into frictional contact with an interior surface of the guide channel causing removal of contaminants from the lens.

10. The instrument of claim 9, further comprising a fluid conduit including an outlet for ejection of fluid onto the lens of the scope.

11. The instrument of claim 9, further comprising a fluid conduit in fluid communication with the cleaner.

12. The instrument of claim 8, wherein the guide channel is centrally disposed within the lumen of the sheath.

13. The instrument of claim 1, wherein a proximal portion of the cleaner is moister than a distal portion of the cleaner.
